(19) United States
(12) Patent Application Publication
(10) Pub. No.: US 2019/0365555 A1
(43) Pub. Date: Dec. 5, 2019

(54) Orthopedic Wrist Device

(71) Applicant: Genti Kamberi, Mississauga (CA)

(72) Inventor: Genti Kamberi, Mississauga (CA)

(21) Appl. No.: 15/993,131

(22) Filed: May 30, 2018

(51) Int. Cl. A61F 5/01

(52) U.S. Cl. CPC A61F 5/0118; A61F 2005/0155; A61F 5/013

(57) ABSTRACT

An orthopedic wrist device for stabilizing a wrist includes a panel that is flexible. Each of a pair of straps is coupled to the panel proximate to a respective opposing end of the panel and extends from each opposing side of the panel. Each of a pair of first couplers and each of a pair of second couplers are coupled to a respective strap proximate to a first end and a second end of the respective strap, respectively. The straps are configured to position around a wrist of a user so that the panel is positioned on an underside of the wrist. Each second coupler is positioned to selectively couple to an associated first coupler to removably couple the panel to the wrist to stabilize the wrist. A base is coupled to and extends from a bottom of the panel and is configured to support the wrist on a surface.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to orthopedic devices and more particularly pertains to a new orthopedic device for stabilizing a wrist.

### BRIEF SUMMARY OF THE INVENTION

An embodiment of the disclosure meets the needs presented above by generally comprising a panel that is flexible. Each of a pair of straps is coupled to the panel proximate to a respective opposing end of the panel and extends from each opposing side of the panel. Each of a pair of first couplers and each of a pair of second couplers are coupled to a respective strap proximate to a first end and a second end of the respective strap, respectively. The straps are configured to position around a wrist of a user so that the panel is positioned on an underside of the wrist. Each second coupler is positioned to selectively couple to an associated first coupler to removably couple the panel to the wrist to stabilize the wrist. A base is coupled to and extends from a bottom of the panel and is configured to support the wrist on a surface.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of an orthopedic wrist device according to an embodiment of the disclosure.

FIG. 2 is an exploded view of an embodiment of the disclosure.

FIG. 3 is a side view of an embodiment of the disclosure.

FIG. 4 is a cross-sectional view of an embodiment of the disclosure.

FIG. 5 is a bottom view of an embodiment of the disclosure.

FIG. 6 is an in-use view of an embodiment of the disclosure.

### DETAILED DESCRIPTION OF THE INVENTION

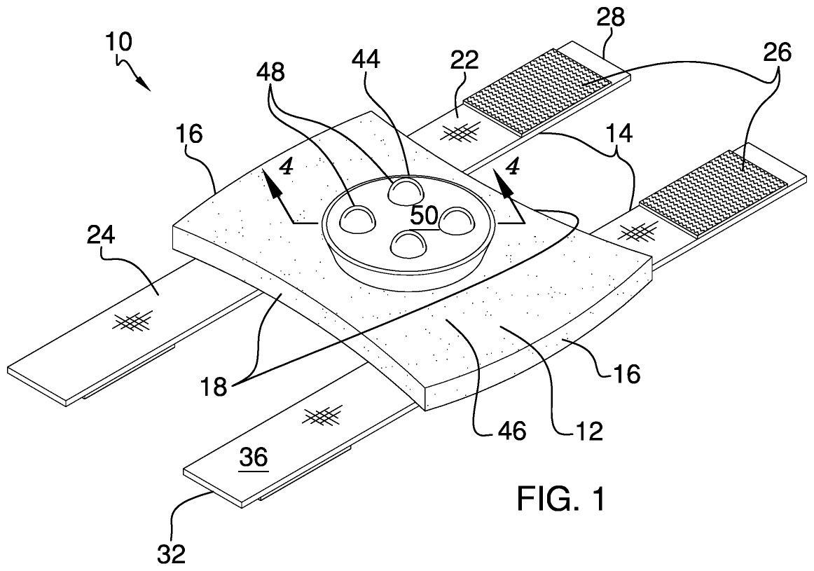

With reference now to the drawings, and in particular to FIGS. 1 through 6 thereof, a new orthopedic device embodying the principles and concepts of an embodiment of the disclosure and generally designated by the reference numeral 10 will be described.

As best illustrated in FIGS. 1 through 6, the orthopedic wrist device 10 generally comprises a panel 12. The panel 12 is flexible. The panel 12 comprises neoprene or the like.

Each of a pair of straps 14 is coupled to the panel 12 proximate to a respective opposing end 16 of the panel 12 so that the strap 14 extends from each opposing side 18 of the panel 12. The straps 14 are positioned on a top 20 of the panel 12. The straps 14 comprise elastic, or the like, so that the straps 14 are resilient. Each strap 14 comprises a first strip 22 and a second strip 24. The first strip 22 and the second strip 24 are coupled singly to and extend from the opposing sides 18 of the panel 12.

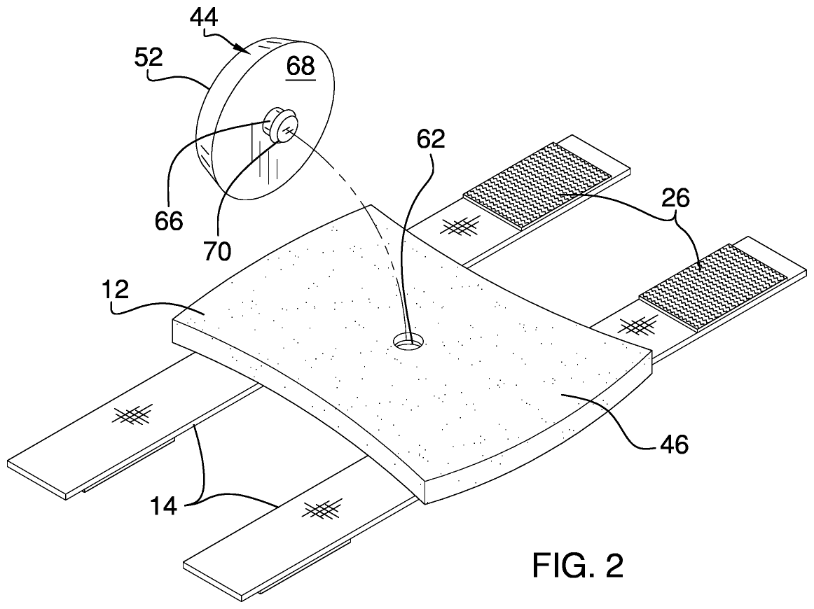

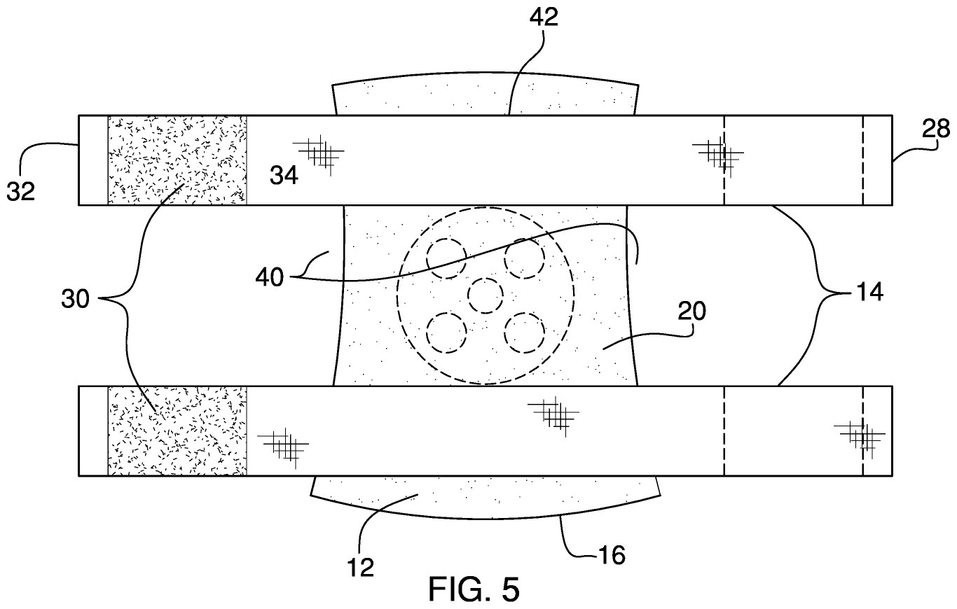

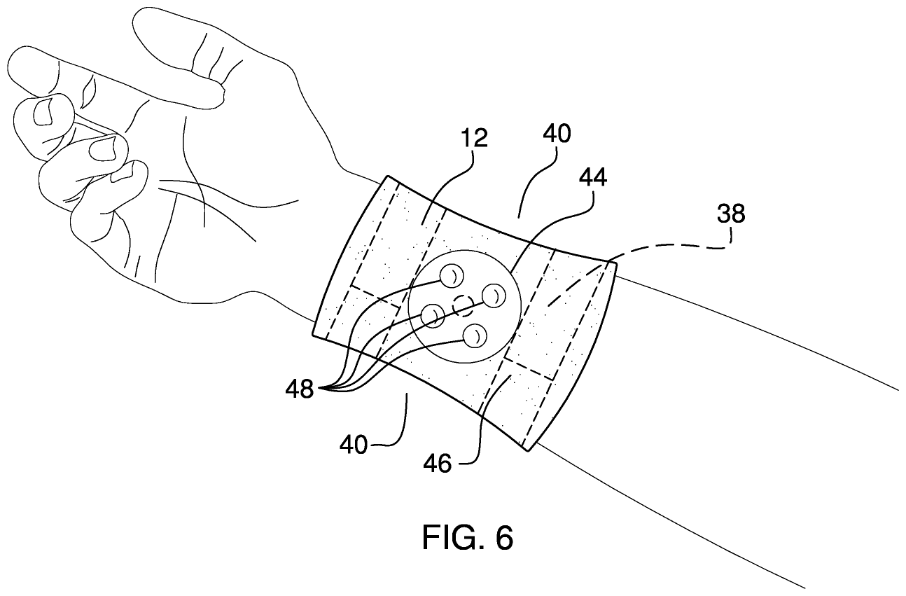

Each of a pair of first couplers 26 is coupled to a respective strap 14 proximate to a first end 28 of the respective strap 14, as shown in FIG. 2. Each of a pair of second couplers 30 is coupled to a respective strap 14 proximate to a second end 32 of the respective strap 14, as shown in FIG. 5. The second couplers 30 are complementary to the first couplers 26. The straps 14 are configured to position around a wrist of a user, positioning the panel 12 on an underside of the wrist, as shown in FIG. 6. Each second coupler 30 is positioned to selectively couple to an associated first coupler 26 to removably couple the panel 12 to the wrist to stabilize the wrist. The second coupler 30 is positioned on an upper surface 34 of the strap 14. The associated first coupler 26 is positioned on a lower surface 36 of the strap 14. Each second coupler 30 and the associated first coupler 26 comprise a hook and loop fastener 38.

Each of a pair of cutouts 40 extends arcuately into a respective opposing side 18 of the panel 12, as shown in FIG. 5. The respective opposing end 16 of the panel 12 is arcuate and extends beyond an edge 42 of the strap 14. The cutouts 40 and the opposing ends 16 complement a shape of the underside of the wrist.

A base 44 is coupled to and extends from a bottom 46 of the panel 12, as shown in FIG. 1. The base 44 is configured to support the wrist on a surface. For example, with the panel 12 coupled to the wrist of the user, the base 44 is positioned approximately 2.5 centimeters toward an elbow from the wrist. This positioning allows the base 44 to support the wrist on the surface with fingers of the hand extending to a keyboard. The base 44 is disc shaped. The base 44 is substantially centrally positioned on the bottom 46 of the panel 12. The base 44 is rigid. The base 44 comprises plastic or the like.

A plurality of rollers 48 is coupled to a second face 50 of the base 44. The rollers 48 are configured to facilitate locomotion of the wrist of the user along the surface. The rollers 48 ensure optimal mobility by providing an easy sliding motion of the wrist along the surface, as between the keyboard and a mouse. The plurality of rollers 48 comprises four rollers 48 that are substantially evenly spaced proximate to a perimeter 52 of the base 44.

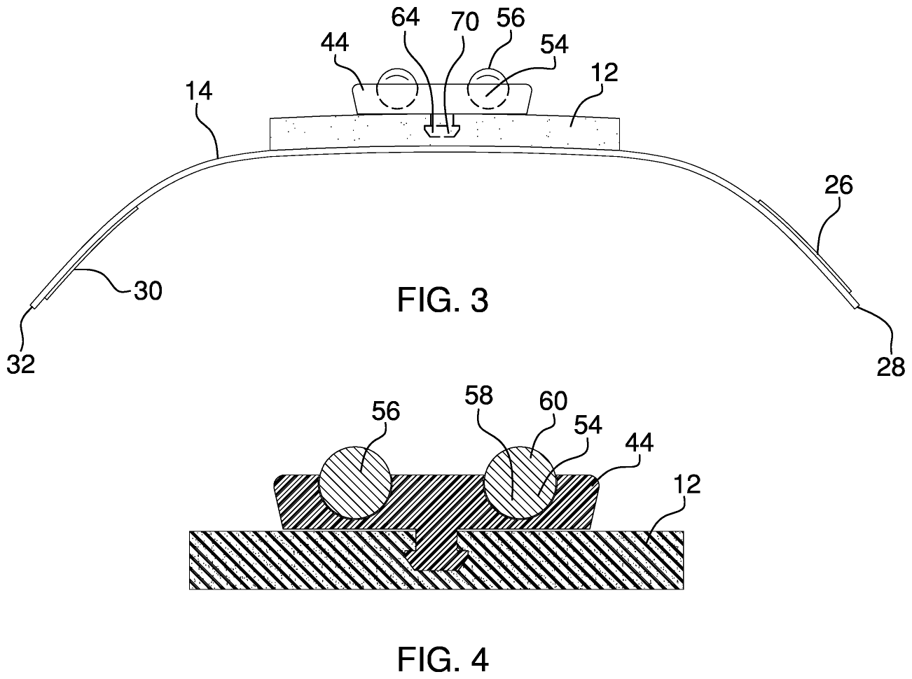

Each roller 48 comprises a recess 54 and a ball 56, as shown in FIG. 3. The recess 54 extends into the base 44 from the second face 50 of the base 44. The recess 54 is shaped complementarily to a first section 58 of the ball 56. The ball 56 is positioned in the recess 54 so that a second section 60 of the ball 56 protrudes from the second face 50. The first section 58 of the ball 56 is larger than the second section 60, as shown in FIG. 2, so that the ball 56 is rotatably coupled to the base 44. The ball 56 is positioned to rotate relative to the base 44 to facilitate the locomotion of the wrist of the user along the surface.

A channel 62 extends into the bottom 46 of the panel 12, as shown in FIG. 2. The channel 62 is circumferentially larger distal from the bottom 46 to define a pocket 64 within the panel 12. A post 66 is coupled to and extends from a first face 68 of the base 44. A tab 70 is coupled to the post 66 distal from the base 44. The post 66 and the tab 70 are shaped complementary to the channel 62. The post 66 and the tab 70 are positioned to be inserted into the channel 62 so that the tab 70 reversibly locks into the pocket 64 to removably couple the base 44 to the panel 12. The ability to remove the base 44 from the panel 12 allows the user to work on a laptop computer with the base 44 removed.

In use, the straps 14 are positioned around the wrist of the user, positioning the panel 12 on an underside of the wrist. Each second coupler 30 is coupled to the associated first coupler 26 to couple the panel 12 to the wrist. The panel 12 stabilizes the wrist and the base 44 supports the wrist. The rollers 48 facilitate the sliding motion of the wrist between the keyboard and the mouse.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An orthopedic wrist device comprising:
a panel, the panel being flexible;
a pair of straps, each strap being coupled to the panel proximate to a respective opposing end of the panel such that the strap extends from each opposing side of the panel;
a pair of first couplers, each first coupler being coupled to a respective strap proximate to a first end of the respective strap;
a pair of second couplers, each second coupler being coupled to a respective strap proximate to a second end of the respective strap, the second couplers being complementary to the first couplers wherein the straps are configured for positioning around a wrist of a user positioning the panel on an underside of the wrist such that each second coupler is positioned for selectively coupling to an associated first coupler for removably coupling the panel to the wrist for stabilizing the wrist; and
a base coupled to and extending from a bottom of the panel wherein the base is configured for supporting the wrist on a surface.

2. The device of claim 1, further including the panel comprising neoprene.

3. The device of claim 1, further including the straps being positioned on a top of the panel.

4. The device of claim 1, further including the straps comprising elastic such that the straps are resilient.

5. The device of claim 1, further including each strap comprising a first strip and a second strip, the first strip and the second strip being coupled singly to and extending from the opposing sides of the panel.

6. The device of claim 1, further including the associated first coupler being positioned on a lower surface of the strap, the second coupler being positioned on an upper surface of the strap.

7. The device of claim 6, further including each second coupler and the associated first coupler comprising a hook and loop fastener.

8. The device of claim 1, further including the base being disc shaped.

9. The device of claim 1, further including the base being substantially centrally positioned on the bottom of the panel.

10. The device of claim 1, further including the base being rigid.

11. The device of claim 10, further including the base comprising plastic.

12. The device of claim 1, further comprising:
a channel extending into the bottom of the panel, the channel being circumferentially larger distal from the bottom defining a pocket within the panel;
a post coupled to and extending from a first face of the base; and
a tab coupled to the post distal from the base such that the post and the tab are shaped complementary to the channel wherein the post and the tab are positioned for inserting into the channel such that the tab reversibly locks into the pocket for removably coupling the base to the panel.

13. The device of claim 10, further including a plurality of rollers coupled to a second face of the base wherein the rollers are configured for facilitating locomotion of the wrist of the user along the surface.

14. The device of claim 13, further including the plurality of rollers comprising four rollers substantially evenly spaced proximate to a perimeter of the base.

15. The device of claim 13, further including each roller comprising a recess and a ball, the recess extending into the base from the second face of the base, the recess being shaped complementarily to a first section of the ball, the ball being positioned in the recess such that a second section of the ball protrudes from the second face, the first section of the ball being larger than the second section such that the ball is rotatably coupled to the base wherein the ball is positioned for rotating relative to the base for facilitating the locomotion of the wrist of the user along the surface.

16. The device of claim 1, further including a pair of cutouts, each cutout extending arcuately into a respective opposing side of the panel.

17. The device of claim 1, further including the respective opposing end of the panel being arcuate and extending beyond an edge of the strap.

18. An orthopedic wrist device comprising:
a panel, the panel being flexible, the panel comprising neoprene;
a pair of straps, each strap being coupled to the panel proximate to a respective opposing end of the panel such that the strap extends from each opposing side of the panel, the straps being positioned on a top of the panel, the straps comprising elastic such that the straps are resilient, each strap comprising a first strip and a second strip, the first strip and the second strip being coupled singly to and extending from the opposing sides of the panel;
a pair of first couplers, each first coupler being coupled to a respective strap proximate to a first end of the respective strap;
a pair of second couplers, each second coupler being coupled to a respective strap proximate to a second end of the respective strap, the second couplers being complementary to the first couplers wherein the straps are configured for positioning around a wrist of a user positioning the panel on an underside of the wrist such that each second coupler is positioned for selectively coupling to an associated first coupler for removably coupling the panel to the wrist for stabilizing the wrist, the second coupler being positioned on an upper surface of the strap and the associated first coupler being positioned on a lower surface of the strap, each second coupler and the associated first coupler comprising a hook and loop fastener;
a base coupled to and extending from a bottom of the panel wherein the base is configured for supporting the wrist on a surface, the base being disc shaped, the base being substantially centrally positioned on the bottom of the panel, the base being rigid, the base comprising plastic;
a channel extending into the bottom of the panel, the channel being circumferentially larger distal from the bottom defining a pocket within the panel;
a post coupled to and extending from a first face of the base;
a tab coupled to the post distal from the base such that the post and the tab are shaped complementary to the channel wherein the post and the tab are positioned for inserting into the channel such that the tab reversibly locks into the pocket for removably coupling the base to the panel;
a plurality of rollers coupled to a second face of the base wherein the rollers are configured for facilitating locomotion of the wrist of the user along the surface, the plurality of rollers comprising four rollers substantially evenly spaced proximate to a perimeter of the base, each roller comprising a recess and a ball, the recess extending into the base from the second face of the base, the recess being shaped complementarily to a first section of the ball, the ball being positioned in the recess such that a second section of the ball protrudes from the second face, the first section of the ball being larger than the second section such that the ball is rotatably coupled to the base wherein the ball is positioned for rotating relative to the base for facilitating the locomotion of the wrist of the user along the surface; and
a pair of cutouts, each cutout extending arcuately into a respective opposing side of the panel, the respective opposing end of the panel being arcuate and extending beyond an edge of the strap.
